(19) United States
(12) Patent Application Publication
(10) Pub. No.: US 2025/0085269 A1
(43) Pub. Date: Mar. 13, 2025

(54) Quantitative Characterization of Curly Hair Phenotypes

(71) Applicants: Spelman College, Atlanta, GA (US); University of Massachusetts Amherst, Hadley, MA (US)

(72) Inventors: Michelle Gaines, Atlanta, GA (US); Imani Page, Hadley, MA (US); Nolan Miller, Hadley, MA (US); Alfred Crosby, Hadley, MA (US); Benjamin Greenvall, Hadley, MA (US); Gregory Grason, Hadley, MA (US)

(21) Appl. No.: 18/883,405

(22) Filed: Sep. 12, 2024

Related U.S. Application Data
(60) Provisional application No. 63/582,147, filed on Sep. 12, 2023.

(51) Int. Cl. G01N 33/483; G01N 3/08

(52) U.S. Cl. CPC G01N 33/4833; G01N 3/08; G01N 2203/0017; G01N 2203/0075; G01N 2203/0094; G01N 2203/028

(57) ABSTRACT

This disclosure provides new and improved processes and methods for quantitative analysis and classification of hair phenotypes, specifically curly hair phenotypes. In an aspect, quantitative geometric measurements can be used to determine whether a hair fiber is wavy, curly, coiled, kinky, or kinky-coiled.

### CROSS-REFERENCE TO RELATED APPLICATIONS

This application claims priority to and the benefit of U.S. Provisional Application No. 63/582,147, filed on Sep. 12, 2023, which is hereby incorporated by reference in its entirety.

### GOVERNMENT SUPPORT

This invention was made with government support under grant number 2137578 awarded by the U.S. National Science Foundation. The government has certain rights in the invention.

### TECHNICAL FIELD

This disclosure relates to processes and methods of analyzing and classifying hair phenotypes.

### BACKGROUND

Hair is a natural polymeric composite that is primarily composed of tight macro-bundles of keratin proteins, which are highly responsive to external stimuli, including pH, temperature, and ionic solvent content. The external responsive behavior displayed by hair is similar to the behavior displayed by hydrogels and other natural fibrous gel systems like collagen and fibrin.

Hair and its appearance also play a significant role in human society by serving as a form of nonverbal social expression. It is a highly complex biocomposite system, which has been traditionally challenging to characterize and thus develop functional personal care products for consumers. Over the last few decades, a significant social paradigm shift occurred among those with curly hair, as they began to accept the natural morphological shape of their curls and style their hair according to its innate, distinct, and unique material properties. These societal and cultural shifts have given rise to the development of new hair classification systems, beyond the traditional and highly limited race-based distinctions.

For example, one hair typing taxonomy based on quantitative geometric parameters displayed among the four key curl patterns-straight, wavy, curly, and coiled—was developed. However, this system fails to capture the complex diversity of curly and coiled hair. Subsequently, another classification was developed that is the existing gold standard for classifying curl and coiled hair, however this system relies upon qualitative classification measures, making the system vague and ambiguous and to the full diversity of phenotypic differences.

While hair science has greatly advanced in recent decades, there is still a need for products that protect the natural mechanics of hair strands.

Therefore, there remains a need for developing new and improved processes and methods for quantitative analysis and classification of hair phenotypes, specifically curly hair phenotypes.

### SUMMARY OF THE DISCLOSURE

In an aspect, this disclosure provides new and improved processes and methods for quantitative analysis and classification of hair phenotypes.

According to aspects, this disclosure provides a method of classifying hair, where the method includes the steps of obtaining quantitative measurements of at least one mechanical property, at least one geometric property, or both, of a test sample of hair, comparing the quantitative measurements of the test sample with the corresponding quantitative measurements of a plurality of reference samples of hair, each reference sample having a different phenotype, and assigning a phenotype to the test sample based upon a closest correlation of quantitative measurements of the test sample and quantitative measurements of the plurality of reference samples.

According to other aspects, this disclosure provides a method of selecting a hair treatment product, where the method includes the steps of obtaining quantitative measurements of at least one mechanical property, at least one geometric property, or both, of a test sample of hair, comparing the quantitative measurements of the test sample with the corresponding quantitative measurements of a plurality of reference samples of hair, each reference sample having a different phenotype, assigning a phenotype to the test sample based on a closest correlation of quantitative measurements of the test sample and quantitative measurements of the plurality of reference samples, and selecting a hair treatment product comprises at least one ingredient selected to achieve a desired effect on the assigned phenotype of the test sample of hair.

According to further aspects, this disclosure provides a method for determining a hair phenotype, where the method includes the steps of obtaining quantitative measurements of at least one geometric property of a test sample of hair, wherein the at least one geometric property is selected from contour length (L), a pitch (ρ), a number of contours in a unit length, or any combination thereof, comparing the quantitative measurements of the test sample with a reference guide comparing quantitative measurements of the at least one geometric property with hair phenotype, and assigning a phenotype to the test sample based upon the closest correlation of quantitative measurements of the test sample and the reference guide.

These and other features, embodiments and aspects of the processes and methods are described more fully in the Detailed Description and claims and further disclosure such as the Examples provided herein.

### BRIEF DESCRIPTION OF THE DRAWINGS

FIGS. 1A-1C. Photogrammetry. (a) Experimental design for capturing data for 3D rendering of hair fibers. (b) 3D render of curly hair fiber mounted to the Texture Analyzer (TA). The fiber was imaged at zero displacement. (c) Fiber length measured in Blender (3D-modeling software) by tracing the path along the length rendered 3D curve. Curve length of pictured fiber 37.519+0.454 mm.

FIGS. 2A-2H. Hair Fiber Ellipticity & Hair Diameter Measurements. Bright-field optical microscopy and SEM images of hair fibers. (a-c) Average diameter (d) was measured from 3-5 strands of each hair sample, along multiple points along the length of each fiber. The samples shown are (a) straight, (b) wavy, and (c) curly hair fibers. According to Walker's taxonomy, the samples would be considered (a) Type-1, (b) Type-2b, and (c) Type-3c. (d) Hair fiber structure. (e-g) scanning electron micrographs of the cross-section of hair samples shown in a-c. Ellipticity (e) is a measure of divergence of an ellipse from a circle. It is the ratio of lengths of the major and minor axes of the cross-section of each hair fiber. The inset (e-g) is an image of each sample of hair fibers vitrified in 1.5 mL vial of epoxy resin. Images were collected of each cross-section after clipping the tip of each sample vial. The scale bar in (a) and (c) is 50 μm, (b) is 80 μm, and (g) is 200 μm. The scale bar in the inset of (g) is 10 mm. (h) Summary table comparing average diameter to average ellipticity for each curly type.

FIG. 3. Summary of New Geometric Measurements. Summary of measurements collected on samples of straight (1), wavy (2a-c), curly (3a-c), and kinky (4a-c) hair. Measurements reported are the average taken from measurements collected off 12-15 strands from each sample of cleaned, dried hair fibers. Samples c1 and c2 signify 2 different samples from the same curl type.

FIG. 4. Contour Length, Number of Contours, & Pitch Geometry Parameters. The three key parameters measured are L, No. of Contours, & p. Contour Length (L)-length in centimeters along one contour of a wavy or curly hair fiber. Number of Contours in 3 cm (No. of Contours)-number of L within a 3 cm distance, measured parallel to the direction of hair growth. Pitch (p)-distance between the start and the end of one L, measured parallel to the direction of hair growth.

FIG. 5. Graphical representations of new parameters, No. of Contours & (3/L)/p, were identified and compared against Andre Walker's hair-typing system. (Left) No. of Contours represents the number of L within a 3 cm distance, measured parallel to the direction of hair growth. (Right) (3/L)/p is a derivative of curl index (i), which describes the extent of curling observed in curly and kinky hair. (3/L)/p requires quantitative measures to determine (Right). Measurements correlate evenly with results, where no. of contours was estimated manually by counting number of L observed against a ruler (3 cm) (Left).

FIGS. 6A-6C. (a) Example stress-strain graph for curly or kinky hair fibers. I-Toe Region, the tensile force required to uncurl the natural curve morphology of a curly and kinky hair fiber (σᵤ). The toe region is virtually absent for straight and wavy hair samples (Types 1 & 2). II-Elastic region, used to determine the elastic modulus of the hair strand. III-Plastic Region, plastic deformation, permanently stretching the hair past its maximum tensile stress (σₘₐₓ) thus causing permanent damage to the cortex. IV-Fracture Force, the maximum force the hair strand can endure before fracture. (b) Stress-strain data of hair samples. (Top) compares mechanical behavior of wavy hair against a curly 3c sample. (Bottom) compares mechanical behavior of kinky hair against a curly 3c sample. (c) Stretch ratio highlighting Region I in hair samples. (Top) compares stretch ratio of wavy hair against a curly 3c sample. (Bottom) compares a stretch ratio of kinky hair against a curly 3c sample.

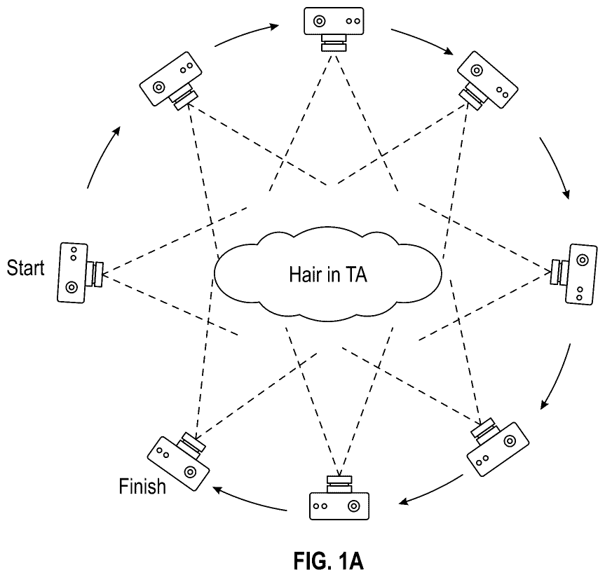

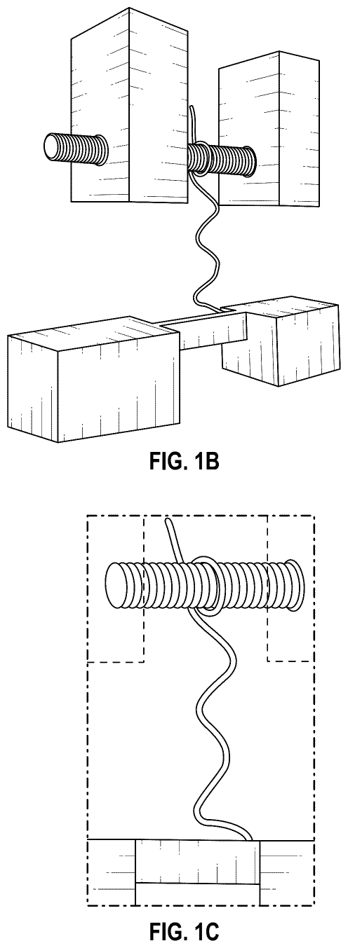

### DETAILED DESCRIPTION

To define more clearly the terms used herein, the following definitions are provided and, unless otherwise indicated or the context requires otherwise, these definitions are applicable throughout this disclosure. If a term is used in this disclosure but is not specifically defined herein, the definition from the IUPAC Compendium of Chemical Terminology, 2nd Ed (1997) can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

When numerical ranges are disclosed, Applicant's intent is to disclose or claim individually each number that such a range could reasonably encompass, including end points of the range as well as any sub-ranges and combinations of sub-ranges encompassed therein, unless otherwise specified. For example by disclosing a length of 1 cm to 10 cm, Applicant's intent is to recite individually 1 cm, 2 cm, 3 cm, 4 cm, 5 cm, 6 cm, 7 cm, 8 cm, 9 cm, and 10 cm, including any sub-ranges and combinations of sub-ranges encompassed therein, and these methods of describing such ranges are interchangeable. Applicant reserves the right to proviso out any individual values, ranges, or sub-ranges that can be claimed according to a range, if for any reason Applicant chooses to claim less than the full measure of the disclosure.

Values or ranges may be expressed using the term “about”, and when such values or ranges are expressed, other embodiments disclosed include the specific value recited, from the one particular value, and/or to the other particular value. In one aspect, use of the term “about” can be replaced with ±15% of the stated value, ±10% of the stated value, or ±5% of the stated value.

In an aspect, this disclosure provides new and improved methods for classifying hair, where the classification is based on quantitative methods utilizing new geometric parameters and material properties that account for more phenotypic differences in curly and kinky hair. In some aspects, the classification system may be used to guide formulation and selection of personal care products that best resonate with curl pattern to maximize desired appearance and overall hair health. In other aspects, the classification system may allow people to objectively classify their own hair. In further aspects, the classification system may be used to assist in product development and creating products specifically for different hair phenotypes.

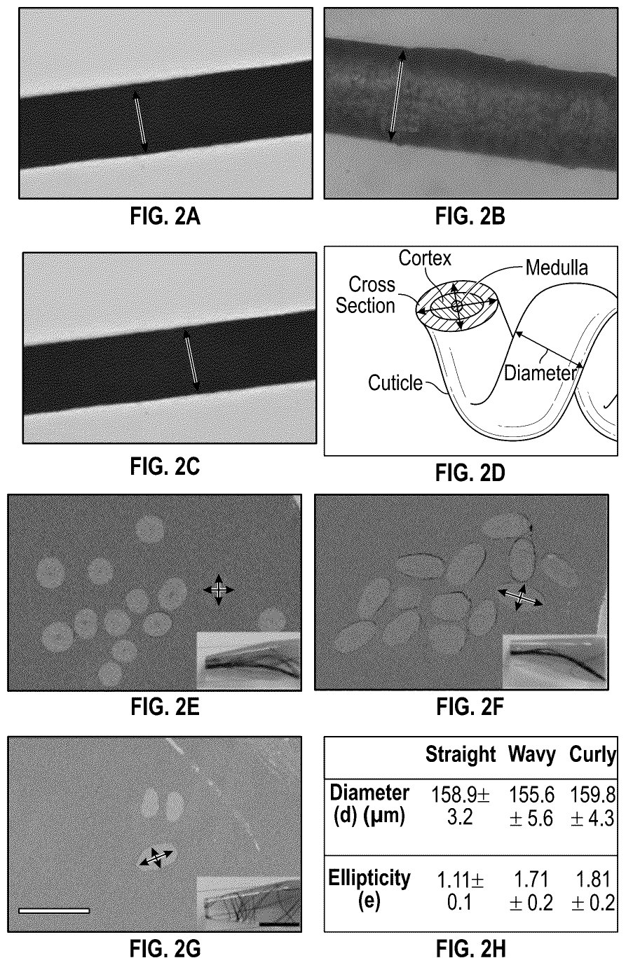

Hair is generally composed of three main components—the medulla, the cortex, and the cuticle (FIG. 2D). The core of each hair fiber consists of the medulla, which is structurally similar to bone marrow, however it is predominantly present in only coarser hair fibers. The cortex surrounds the medulla and provides the bulk of geometric structure, morphological shape, mechanical strength, and elasticity for hair fibers. The cortex is composed of cortical anisotropic cells among a matrix of keratinized amorphous proteins. Each cortical cell is a macro-fibril composite of aggregated alpha-helical micro-fibrils. The inner-structure of micro-fibrils is made of keratin proteins, arranged in tight bundles of alpha-helical keratein fiber intermediate filaments that have been aggregated and arranged into micro-alpha-helices.

Human hair contains three types of cortical cells—paracortical, mesocortical, and orthocortical—each differing slightly in shape. The structural arrangement of each type of cortical cell directly correlates to hair fiber shape, which originates from di-sulfide bond chemical crosslinking between cortical cells, keratinous amorphous proteins, and the long-range order of the resulting cross-linked structures. Lateral phase segregation between the three cortical cells may lead to curl formation in hair, implying more homogenous cortical cell arrangements with wavy and straight phenotypes.

The outer layer of hair—the cuticle—protects the cortex from outside physical and chemical stresses and maintains a homeostatic internal environment. The cuticle is comprised of overlapping layers of cell sheaths that are individually anchored to the base of the cortex and arranged over each other like shingles on a roof. Each cell sheath is composed of keratinized amorphous proteins and several types of lipids. The cuticle is made of five to eleven layers of sheathed cells, which can lift and lower in response to the hair fiber's environment. The natural degradation of cuticle sheaths coupled with their ability to reversibly lift away from the cortex when exposed to changes in humidity and moisture creates porosity within the hair. Hair porosity is defined as the extent of reversible sheath cell opening and closing. Thus, the cuticle is responsible for maintaining hydration across each hair fiber.

While these and other measurements may be helpful in determining hair phenotypes, existing methods of analysis are not easily accessible to the ordinary curly-haired individual. However, certain quantitative measurements that require little to no equipment, such that these measurements may be taken by the average person without access to specialized lab equipment so that they may more accurately determine their hair phenotype.

For example, fiber geometry (length, diameter, ellipticity, and degree of curl), emergence angle from the scalp, and fiber-to-fiber interactions may be key variables to consider when studying hair and hair phenotypes. For example, curlier hair have shorter lengths, may be less dense, and may have slower growth rates. As for the other geometrical parameters, diameter (d) may be used as a geometric descriptor to infer coarseness and thus “texture”, “handle”, and “feel” of the hair. Diameter (d) may also be used to draw conclusions on fiber growth rate and trends in hair fiber density to evaluate scalp health.

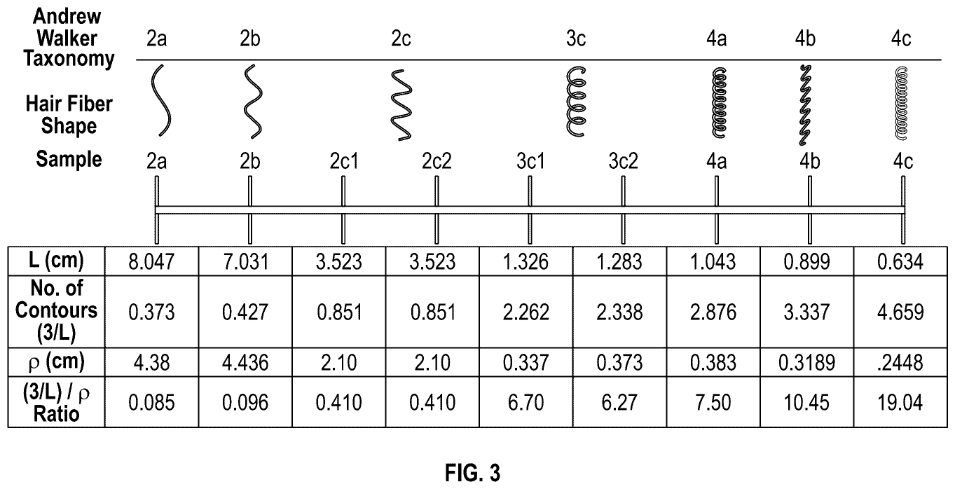

However, there may be several limitations when using diameter (d) to differentiate hair fiber geometry across phenotypes. For example, the diameter (d) of hair fibers may vary significantly from the root to tip, and the diameter (d) of hair fibers may also vary across each person's scalp. Importantly, diameter (d) does not capture ellipticity (e) of each hair fiber, which is a measure of divergence of an ellipse from a circle, i.e., the ratio of lengths of the major and minor axes of the cross-section of each hair fiber (FIG. 3). Ellipticity (e) is a normalized value, and may be used to examine structural differences along the hair shaft in curly hair. Ellipticity (e) can also be an indication of the “degree of curl,” although there may be additional contributors to the degree of curl, such as cortical cell density and distribution, each of which may play a role in curl morphology.

Additional geometrical parameters therefore may be useful in identifying morphological differences between hair types, particularly between curly hair types. For example, contour length (L), number of contours (3/L), pitch (ρ), and number of contours/pitch ratio ((3/L)/ρ) may be directly correlated to hair phenotypes, i.e., wavy, curly, coiled, kinky, or kinky-coiled hair. Advantageously, these parameters may be easily measured without specialized technology or equipment, making more precise hair phenotyping more accessible to the public.

In aspects, the classification system permits a curly-haired individual to identify their hair phenotype. In one aspect, the method of classifying hair includes the steps of:
(a) obtaining quantitative measurements of at least one mechanical property, at least one geometric property, or both, of a test sample of hair;
(b) comparing the quantitative measurements of the test sample with the corresponding quantitative measurements of a plurality of reference samples of hair, each reference sample having a different phenotype; and
(c) assigning a phenotype to the test sample based upon a closest correlation of quantitative measurements of the test sample and quantitative measurements of the plurality of reference samples.

In other aspects, the method of classifying hair includes the steps of:
(a) obtaining quantitative measurements of at least one geometric property of a test sample of hair, wherein the at least one geometric property is selected from contour length (L), a pitch (ρ), a number of contours in a unit length (3/L), or any combination thereof;
(b) comparing the quantitative measurements of the test sample with a reference guide comparing quantitative measurements of the at least one geometric property with hair phenotype;
(c) assigning a phenotype to the test sample based upon the closest correlation of quantitative measurements of the test sample and the reference guide.

The phenotype may be a straight phenotype, a wavy phenotype, a curly phenotype, a coiled phenotype, a kinky phenotype, or a kinky-coiled phenotype.

The wavy phenotype may be generally characterized by hair fibers that have a loose “S” shape. Wavy hair phenotypes may have various levels of definition, such as loose waves, moderately defined waves, and defined waves. Loose waves may lack volume and definition, whereas defined waves are voluminous and have a well-defined loose “S” shape. Moderately defined waves may lack definition near the root, but become more well-defined towards the tips of the hair fibers.

The curly phenotype may be generally characterized by hair fibers that have spiral or helix shape or pattern. Curly hair phenotypes may include loose spiral curls, defined curls, and corkscrew curls. Loose spiral curls may be softer and more rounded in appearance, where defined and corkscrew curls may be more tightly coiled and well-defined. Corkscrew curls may be more tightly coiled than defined curls, and those with corkscrew curls may often have thicker hair fibers than those with defined or loose spiral curls.

The coiled phenotype may be generally characterized by hair fibers that are tightly coiled and have a spring-like appearance and/texture. Coiled hair phenotypes may include defined coils, tight defined coils, and tight spiral coils. These hair types are generally similar, in that each type has tightly-coiled spiral curls, with each having different levels of curl definition, i.e., curl tightness.

The kinky phenotype may be generally characterized by hair fibers that are tightly coiled and wiry, with little to no visible curl pattern. Kinky hair phenotypes may include torsion twist kinks, defined kinks, and permanent crimp kinks. Torsion twist kinks may resemble tight spiral coils, but are characterized by a twisting of the hair strand rather than spiraling thereof. Defined kinks may have a visible and well-defined “Z” shaped pattern, while permanent crimp kinks are more tightly bunched in that the individual strands and/or kinks are not easily discernable to the naked eye.

The kinky-coiled phenotype may be generally characterized by hair fibers that share characteristics between kinky and curly hair phenotypes. For example, kinky hair phenotypes may often be considered to be twisted or crimped, whereas curly hair phenotypes may more often be considered to be spirals. Kinky-coiled phenotypes therefore may include a combination of twisted and spiral characteristics of each respective hair type. Thus, the kinky-coiled phenotypes may include torsion twist and spiral kinky curls, defined kinky curls, and permanent crimp and coil kinky curls.

In aspects, the geometric characteristics of a hair fiber may be directly correlated with hair phenotype. In some aspects, a precise phenotype may be determined by measuring and quantifying the geometric characteristics of a hair fiber. In some aspects, a general phenotype may be determined by measuring and quantifying the geometric characteristics of a hair fiber, and visual analysis of said hair fiber may be used to determine the precise phenotype within the more general phenotype category.

In some aspects, wavy hair fibers have the following geometric characteristics. The contour lengths (L) may be greater than about 3 cm, the number of contours in a unit length (3 cm) may be less than 1, the pitch (ρ) may be greater than about 1 cm, and the ratio of number of contours in a unit length to pitch ((3/L)/ρ) may be less than about 1.

In some aspects, curly hair fibers have the following geometric characteristics. The contour lengths (L) may be between about 1 cm to about 5 cm, the number of contours in a unit length (3 cm) may be between about 1 to about 2.5, the pitch (ρ) may be between about 0.5 cm to about 2 cm, and the ratio of number of contours in a unit length to pitch ((3/L)/ρ) may be between about 0.5 to about 7.

In some aspects, coiled hair fibers have the following geometric characteristics. The contour lengths (L) may be less than about 2 cm, the number of contours in a unit length (3 cm) may be between greater than about 2, the pitch (ρ) may be less than about 1 cm, and the ratio of number of contours in a unit length to pitch ((3/L)/ρ) may be greater than about 8.

In some aspects, kinky hair fibers have the following geometric characteristics. The contour lengths (L) may be less than about 1 cm, the number of contours in a unit length (3 cm) may be between greater than about 2.5, the pitch (ρ) may be less than about 1 cm, and the ratio of number of contours in a unit length to pitch ((3/L)/ρ) may therefore be greater than about 10.

In some aspects, kinky-coiled hair fibers have the following geometric characteristics. The contour lengths (L) may be less than about 1 cm, the number of contours in a unit length (3 cm) may be between greater than about 3, the pitch (ρ) may be less than about 1 cm, and the ratio of number of contours in a unit length to pitch ((3/L)/ρ) may be greater than about 15.

In aspects, the hair phenotype may be used to select a hair treatment product having at least one ingredient that is selected to achieve a desired effect on the hair type. Different hair phenotypes may have different needs with regards to suitable hair care products. For example, certain hair phenotypes may be more prone to frizz, which may indicate a lack of moisture being retained in the hair fibers. It therefore may be desirable to treat these hair types with products designed to help hair fibers retain moisture to improve the appearance and health of frizzy hair.

### EXAMPLES

The examples of this disclosure illustrate various quantitative and qualitative analysis of hair samples, and the experimental methods for said analyses.

### Example 1

### Collection and Preparation of Samples

Hair samples were collected from volunteers of varying age (20-50), gender, ethnicity (nationality), and washing and personal care attitudes. The hair samples were collected from fibers that were naturally shed from the scalp after combing, brushing, or other forms of physical manipulation. Each volunteer gave at last twenty strands of hair, 6 cm or longer, to allow for multiple experiments per sample.

Samples were prepared by washing in 1% sodium lauryl sulfate solution with reverse osmosis water for twenty minutes. Next, they were rinsed and placed onto a drying rack to allow curls, waves, or kinks to naturally form as the hair strands dried. Hair strands dried for at least twelve hours before any experimentation.

### Example 2

### Geometric Properties and Fiber Morphology

Geometric properties and fiber morphology were measured and evaluated using bright-field microscopy, scanning electron microscopy (SEM), and photogrammetry. All collected images were analyzed to extract desired parameters by using ImageJ and Blender, free and open-sourced 3D computer graphic modeling software. Photogrammetry was employed to image and reconstruct the full 3D shape of individual hair samples. Samples were imaged via photogrammetry by collecting images of each hair strand from a range of angular perspectives, captured by circling a camera 360° around each hair fiber (FIG. 2). Photogrammetry reconstruction was used to accurately measure the path length (£), as indicated by the red dotted line extending down the length of the hair fiber shaft (FIG. 2B).

Hair fiber diameter (d) was measured from the samples donated by volunteers via bright-field optical microscopy under 20x, 50x, or 63× magnification. The average diameter (d) from those samples (˜ 4 hair strands, per volunteer) are reported in FIG. 3. The results show little variation of the diameter (d) for each main hair shape morphology (straight, wavy, curly).

Ellipticity (e) is a key distinguishing parameter and is measured by the ratio of the lengths of the major and minor axes from the cross-section of each hair strand. Ellipticity (e) was measured from images of hair sample cross-sections collected via SEM. Other newly identified geometric parameters, such as contour length (L), number of contours, and pitch (ρ) were measured from images collected off a standard camera and then analyzed using ImageJ (FIG. 3).

Due to stark differences in dimensionality, it is no surprise that the geometric parameters used to characterize straight and wavy hair may not fully represent the geometric diversity of curly and kinky hair because straight and wavy hair exist in 2-dimensions while other phenotypes exist in 3-dimensions. As a result, new geometrical parameters were reported to capture the morphological differences between wavy, curly, and kinky hair. These new parameters were used to develop a quantitative taxonomy that was compared to existing hair taxonomy systems.

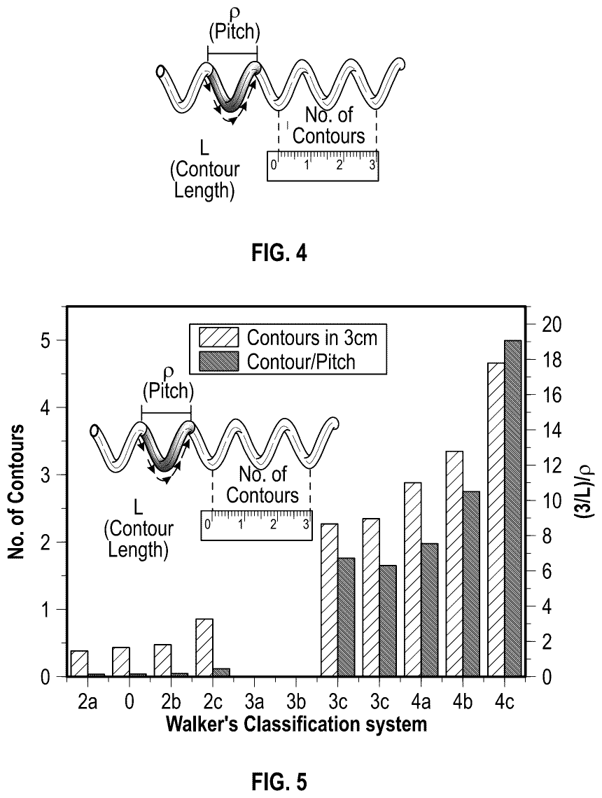

FIG. 4 illustrates the methodology for measuring each of the new geometric variables—contour length (L), number of contours (3/L), pitch (ρ), and number of contours/pitch ratio ((3/L)/ρ). Table 1 below depicts measurable trends for each of the four new geometric parameters across hair phenotypes.

TABLE 1
Summary of New Geometric Measurements. Summary of hair measurements collected
on samples of straight (1), wavy (2a-2c), curly (3a-3c), and kinky (4a-
4c) hair. Measurements reported are the average taken from measurements
collected off 12-15 strands from each sample of cleaned, dried hair fibers.
Sample name is referenced according to the Andre Walker taxonomy.

| Sample | 2a | 2b | 2c1 | 2c2 | 3c1 | 3c2 | 4a | 4b | 4c |
|---|---|---|---|---|---|---|---|---|---|
| L (cm) | 8.047 | 7.031 | 3.523 | 3.523 | 1.326 | 1.283 | 1.043 | 0.899 | 0.634 |
| No. of | 0.373 | 0.427 | 0.851 | 0.851 | 2.262 | 2.338 | 2.876 | 3.337 | 4.659 |
| Contours (3/L) | | | | | | | | | |
| ρ (cm) | 4.38 | 4.436 | 2.10 | 2.10 | 0.337 | 0.373 | 0.383 | 0.3169 | 0.2448 |
| (3/L)/ρ Ratio | 0.085 | 0.096 | 0.410 | 0.410 | 6.7 | 6.27 | 7.5 | 10.45 | 19.04 |

The number of contours was measured per unit length, where the unit length was 3 cm. Thus, the number of contours is represented as (3/L). Measurement were collected from 2D images of clean and dry hair samples using ImageJ. The results show a continuous decrease in the number of contours and a continuous increase in the ratio of contours to pitch.

The relationship between these values is also depicted in FIG. 5, while also being compared with existing hair type classifications. FIG. 5 illustrates overall agreement of the measured results with curl pattern categories and sub-categories. FIG. 5 further illustrates similar trends between quantitative measurements obtained and the trends observed when estimating the number of contours on a strand of hair, by counting the repeating contour lengths (L) observed within 3 cm. The latter methodology allows one to estimate their curl pattern themselves against a ruler, without using any analytical techniques.

Hair samples behave as hypothesized, where the number of contours in 3 cm unit length increases, comparably, with increasingly curly hair phenotypes. Similarly, the ratio of contours to pitch also increases with increasingly curly hair phenotypes.

### Example 3

### Mechanical Properties

Mechanical properties were measured using a texture analyzer (TA) (TA-XT, Stable Microsystems®) and a dynamic mechanical analyzer (DMA). Stress-strain curves were developed from force-displacement measurements collected from each hair strand in the TA. Hair strands were loaded between grips by winding the ends of each strand around the top and bottom grips of the TA and DMA. Before each tensile strength test began, the initial displacement length was set to either 1, 3, or 6 cm, respectively. An example is shown in FIG. 2C, where a 3D rendered curly hair strand is loaded in the TA and set at an initial displacement length of 3 cm. Experiments concluded at the fiber's point of fracture.

It has been previously reported that straight and wavy hair are stronger than curly and kinky hair. It was generally understood that Young's modulus (E), tensile strength (σ), and fracture point decrease with increasing degrees of curliness, which the friction coefficient increases with the degree of curliness. Hair breakage and damage from mechanical manipulation has been widely reported and commonly experienced by people with curly and kinky hair. These conclusions remain true for hair fibers that are dry, wet, or coated with products.

Two tensile forces are known to contribute to the overall strength of hair fibers—uncurling force (σᵤ) and elastic tensile strength (σ_{ε}). Previous works have observed that the overall stress response decreases with increasing hair fiber curliness, meaning that curlier hair fibers exhibit a time-delay before the onset of elastic stress in response to fiber extension (strain). It is also understood that values for σᵤ measured on straight and wavy hair samples are negligible. In curly and kinky hair, fiber viscoelasticity is directly correlated with degree of curliness.

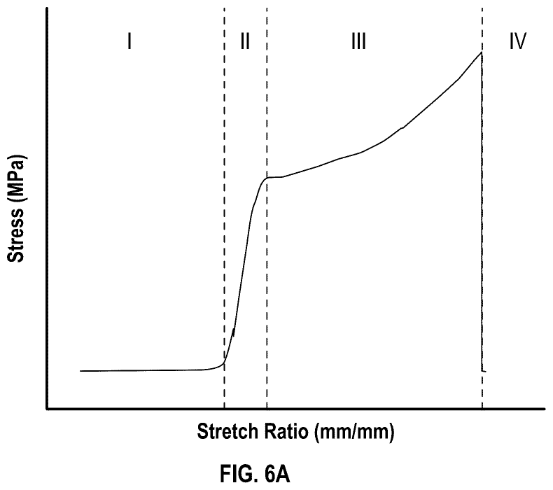

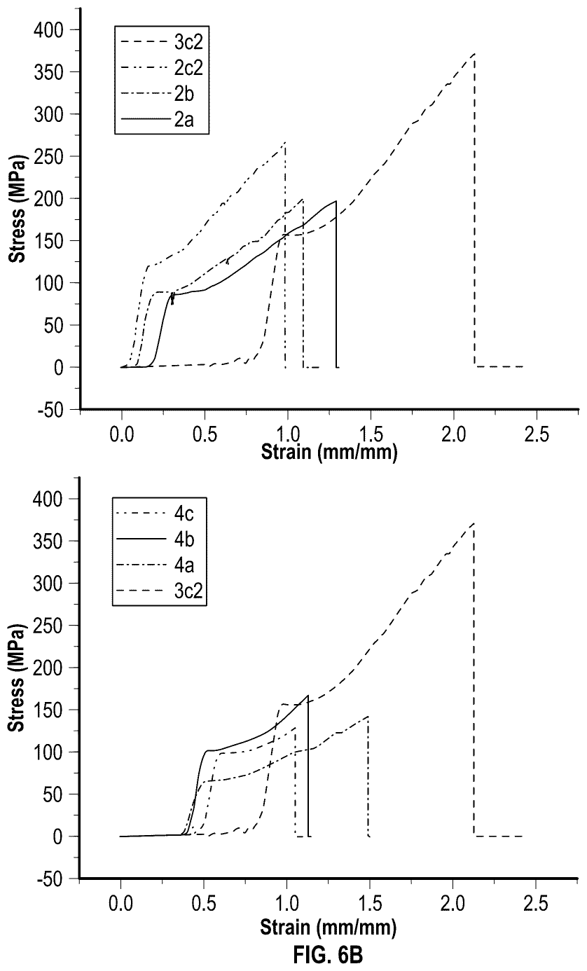

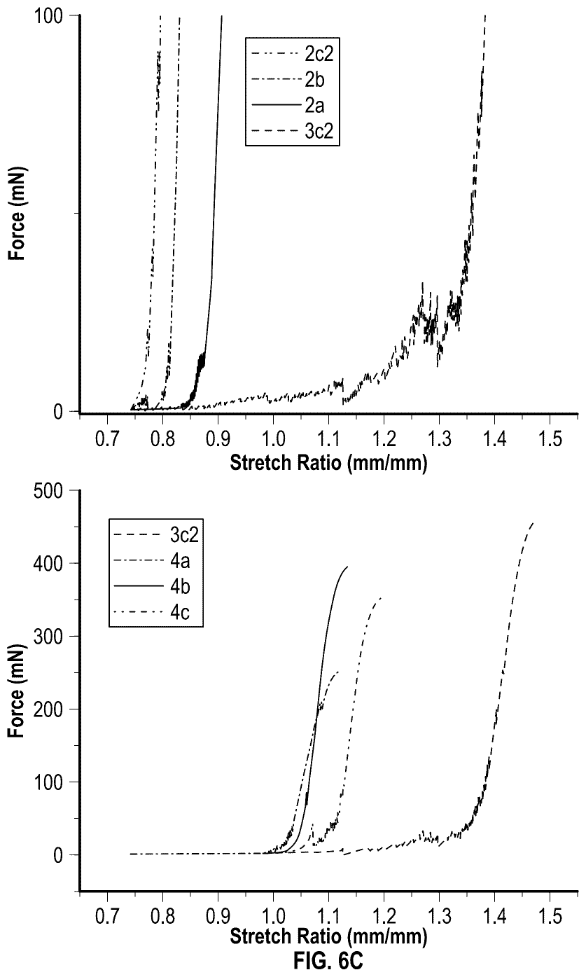

The observed results described further herein depict notable differences in the mechanical response between samples with slight morphological differences in hair fiber geometry. Stress-strain behavior was collected off a texture analyzer (TA) and is summarized in FIG. 6. Region I is the Toe Region, and it describes the stress-strain behavior when a fiber is uncurled (σᵤ). Region II is the elastic region where elastic modulus (E) is determined. Regions II-IV are the regions captured in a typical stress-strain curve for a fiber. DMA can measure mechanical behavior at a higher resolution and was used to measure force displacement response with increased precision. The stress-strain behavior of wavy and curly hair samples shown in FIG. 6B, where the stress-strain behavior of sample 3c was compared against wavy samples (top—2a-2c) and kinky samples (bottom-4a-4c). Sample 3c shows evidence of the widest Toe Region (Region I) and thus the largest σᵤ.

Stretch ratio is a newly studied parameter that further describes the mechanical behavior of hair samples in Region I. Stretch ratio can be calculated using the following equation:

$$\frac{{ε}_{0} + Δ ε}{ℒ} . \qquad ( I )$$

ε₀ is an initial displacement (mm), i.e., the displacement recorded at time=0 seconds. Δε is displacement (mm) and [symbol] is total path length (mm). The stretch ratio depicts the extent of stretching that each hair fiber endures during a tensile test. FIG. 6C compares the extent of hair fiber stretching between a curly 3c sample, wavy hair (2a-2c), and kinky hair (4a-4c). Stretch ratio is 1.0 when ε₀ equals [symbol] , which depicts the mechanical behavior of a straight hair fiber. Wavy hair samples depicted stretch ratios less than 1.0, which generally indicates that [symbol] >ε₀. Each of the kinky hair samples consistently reported [symbol] <ε₀, which captures some extent of initial curliness for each of the hair samples.

That which is claimed is:

1. A method of classifying hair, the method comprising:
obtaining quantitative measurements of at least one mechanical property, at least one geometric property, or both, of a test sample of hair;
comparing the quantitative measurements of the test sample with the corresponding quantitative measurements of a plurality of reference samples of hair, each reference sample having a different phenotype; and
assigning a phenotype to the test sample based upon a closest correlation of quantitative measurements of the test sample and quantitative measurements of the plurality of reference samples.

2. The method of claim 1, wherein the at least one geometric property is selected from a contour length (L), a pitch (ρ), a number of contours in a unit length, a follicle shape, a follicle diameter, or any combination thereof, and wherein the at least one mechanical property is selected from a maximum tensile stress (σₘₐₓ), a stretch ratio, an elastic modulus (E), an uncurling force (σᵤ), an elastic tensile strength (σ_{ε}), or any combination thereof.

3. The method of claim 1, wherein the phenotype is selected from a straight phenotype, a wavy phenotype, a curly phenotype, a coiled phenotype, a kinky phenotype, or a kinky-coiled phenotype.

4. The method of claim 3, wherein the wavy phenotype further comprises a loose wavy phenotype, a moderately defined wavy phenotype, and a defined wavy phenotype.

5. The method of claim 3, wherein the curly phenotype further comprises a loose spiral curly phenotype, a defined curly phenotype, and a corkscrew curly phenotype.

6. The method of claim 3, wherein the coiled phenotype further comprises a defined tight coiled phenotype, a defined coiled phenotype, and a tight spiral coiled phenotype.

7. The method of claim 3, wherein the kinky phenotype further comprises a torsion twist, kinky phenotype, a defined kinky phenotype, and a permanent crimp kinky phenotype.

8. The method of claim 3, wherein the kinky-coiled phenotype further comprises a torsion twist and a spiral kinky-coiled phenotype, a defined kinky-coiled phenotype, and a permanent crimp and coil kinky-coiled phenotype.

9. A method of selecting a hair treatment product, the method comprising:
obtaining quantitative measurements of at least one mechanical property, at least one geometric property, or both, of a test sample of hair;
comparing the quantitative measurements of the test sample with the corresponding quantitative measurements of a plurality of reference samples of hair, each reference sample having a different phenotype;
assigning a phenotype to the test sample based on a closest correlation of quantitative measurements of the test sample and quantitative measurements of the plurality of reference samples; and
selecting a hair treatment product comprises at least one ingredient selected to achieve a desired effect on the assigned phenotype of the test sample of hair.

10. The method according to claim 9, wherein the geometric properties are measured using scanning electron microscopy (SEM), photogrammetry, optical microscopy, or any combination thereof, and wherein the mechanical properties are measured under tensile extension using a texture analyzer (TA), a dynamic mechanical analyzer (DMA), or a combination thereof.

11. The method according to claim 9, wherein the quantitative measurements are selected from a contour length (L), a pitch (ρ), a number of contours in a unit length, a (number of contours in a unit length)/p ratio, a maximum tensile stress (σₘₐₓ), a stretch ratio, the elastic modulus (E), an uncurling force (σᵤ), an elastic tensile strength (σ_{ε}), or any combination thereof.

12. The method according to claim 11, wherein the phenotype is selected from a straight phenotype, a wavy phenotype, a curly phenotype, a coiled phenotype, a kinky phenotype, or a kinky-coiled phenotype.

13. The method according to claim 12, wherein the wavy phenotype is characterized in that the contour length (L) is between about 3.5 cm to about 8 cm, the number of contours in a unit length is less than about 1, the pitch (ρ) is between about 2 cm to about 4.5 cm, and/or the (number of contours in a unit length)/ρ ratio is less than about 1.

14. The method according to claim 12, wherein the curly phenotype is characterized in that the contour length (L) is between about 1 cm to about 3.5 cm, the number of contours in a unit length is between about 1 to about 2.5, the pitch (ρ) is between about 0.5 cm to about 2 cm, and/or the (number of contours in a unit length)/p ratio is between about 0.5 to about 7.

15. The method according to claim 12, wherein the coiled phenotype is characterized in that the contour length (L) is less than about 2 cm, the number of contours in a unit length is greater than about 2, the pitch (ρ) is less than about 1 cm, and/or the (number of contours in a unit length)/p ratio is greater than about 8.

16. The method according to claim 12, wherein the kinky phenotype is characterized in that the contour length (L) is less than about 1 cm, the number of contours in a unit length is greater than about 2.5, the pitch (ρ) is less than about 1 cm, and/or the (number of contours in a unit length)/p ratio is greater than about 10.

17. The method according to claim 12, wherein the kinky-coiled phenotype is characterized in that the contour length (L) is less than about 1 cm, the number of contours in a unit length is greater than about 3, the pitch (ρ) is less than about 1 cm, and/or the (number of contours in a unit length)/p ratio is between greater than about 15.

18. A method for determining a hair phenotype, the method comprising:
obtaining quantitative measurements of at least one geometric property of a test sample of hair, wherein the at least one geometric property is selected from contour length (L), a pitch (ρ), a number of contours in a unit length, or any combination thereof;
comparing the quantitative measurements of the test sample with a reference guide comparing quantitative measurements of the at least one geometric property with hair phenotype;
assigning a phenotype to the test sample based upon the closest correlation of quantitative measurements of the test sample and the reference guide.

19. The method of claim 18, wherein the reference guide is prepared by obtaining quantitative measurements of the at least one geometric property of a plurality of reference samples of hair, each reference sample having a different phenotype.

20. The method of claim 18, wherein the assigned phenotype is used to select a hair treatment product comprising at least one ingredient selected to achieve a desired effect on the assigned phenotype of the test sample of hair.
